(19) United States
(12) Patent Application Publication
(10) Pub. No.: US 2024/0299436 A1
(43) Pub. Date: Sep. 12, 2024

(54) Use of Inosine in Preparing Drug For Treatment of Corona Virus Disease 2019

(71) Applicant: PLA ACADEMY OF MILITARY MEDICAL SCIENCES, Beijing (CN)

(72) Inventors: Yue GAO, Beijing (CN); Wei ZHOU, Beijing (CN); Huipeng CHEN, Beijing (CN); Yuwei GAO, Beijing (CN); Shuchen LIU, Beijing (CN); Yunbo SUN, Beijing (CN); Ningning WANG, Beijing (CN); Entao LI, Beijing (CN)

(21) Appl. No.: 18/271,296

(22) PCT Filed: Mar. 2, 2022
(86) PCT No.: PCT/CN2022/078715

§ 371 Date: Jul. 7, 2023

(30) Foreign Application Priority Data
Dec. 16, 2021 (CN) 202111541575.0

(51) Int. Cl. A61K 31/708

(52) U.S. Cl. CPC A61K 31/708

(57) ABSTRACT

Use of inosine in preparing a drug for the treatment of coronavirus disease 2019 (COVID-19), where a formulation of the drug includes one or more selected from the group consisting of tablets, capsules, oral liquid preparations, emulsions, and injections. Inosine can reduce level of severe acute respiratory syndrome coronavirus 2 (SARS-COV-2) induced interleukin-6 (IL-6), alleviate SARS-COV-2 caused inflammatory lung injury, and further increase the percent survival of critically ill patients with SARS-COV-2.

### CROSS REFERENCE TO RELATED APPLICATION

This patent application claims the benefit and priority of Chinese Patent Application No. 202111541575.0 filed with the China National Intellectual Property Administration on Dec. 16, 2021, and entitled “USE OF DRUG FOR TREATMENT OF CORONA VIRUS DISEASE 2019”, the disclosure of which is incorporated by reference herein in its entirety as part of the present application.

### TECHNICAL FIELD

The present disclosure relates to the technical field of biomedicine, in particular to use of inosine in preparing a drug for treatment of Corona Virus Disease 2019 (COVID-19).

### BACKGROUND

Inosine is the most common hypoxanthine nucleotide substance widely found in related animals and plants. Inosine, as an important drug and a raw material for synthesizing other drugs, involves in various physiological functions and metabolic processes of the organism. In the pharmaceutical industry, inosine can be processed into injections, oral liquids, or tablets. Inosine has excellent cell membrane permeability, which can directly enter cells to involve in material metabolism. Clinically, inosine is indicated for the treatment of symptoms of various acute and chronic liver diseases, heart diseases, leucopenia or thrombocytopenia, central retinitis, and optic atrophy.

Severe acute respiratory syndrome coronavirus 2 (SARS-COV-2) is a novel coronavirus strain that has never been found in humans before. The SARS-COV-2 is a RNA virus, and the genome of which is a linear single-stranded RNA. The virion is round or elliptical. The positive-strand RNA means that when the virus enters a cell, it synthesizes a protein directly and produces negative strands by RNA polymerase for self-replication. The COVID-19 pandemic has been a global pandemic, which has significant adverse effects on human's health and life. So far, there have been more than two hundred million cumulative confirmed cases worldwide, and the viruses are still spreading quickly in some countries.

Immune system overreaction is usually found in a plurality of severe and critically ill patients with SARS-COV-2 infection, which has a significant effect on the percent survival of critically ill patients. It is of great significance to seek a drug for inhibiting SARS-COV-2.

### SUMMARY

An objective of the present disclosure is to provide use of a drug in the treatment of COVID-19. The drug is inosine, which can downregulate high expression of SARS-COV-2 induced interleukin-6 (IL-6) to effectively control SARS-COV-2 caused inflammatory lung injury, further substantially increasing the percent survival of critically ill patients with SARS-COV-2 infection. The drug brings hope to severe and critically ill patients with COVID-19 and their families, and provides a new idea for treatment of severe and critically ill patients with COVID-19.

The foregoing technical objective of the present disclosure is achieved through the following technical solutions:
the present disclosure provides use of inosine in prevention and/or treatment of COVID-19.

The present disclosure further provides use of inosine in preparing drug for prevention and/or treatment of COVID-19.

Preferably, the inosine treats the COVID-19 by inhibiting the secretion of cytokine IL-6 and/or alleviating SARS-COV-2 caused inflammatory lung injury.

Preferably, the treatment of COVID-19 includes increasing the percent survival of treating severe and/or critically ill patients with COVID-19.

Preferably, the inosine includes one or more formulations selected from the group consisting of tablets, capsules, oral liquid preparations, emulsions, and injections.

Preferably, the formulations of drug includes one or more selected from the group consisting of tablets, capsules, oral liquid preparations, emulsions, and injections.

Through the foregoing technical solutions, the inosine may reduce levels of inflammatory factors in patients with autoimmune diseases, cancers or hypersensitivity pneumonitis, inhibit the activation of macrophages, lymphocytes, and neutrophils, and thus improve intestinal and lung injuries. Moreover, the inosine may improve the percent survival of severe and critically ill patients with COVID-19 by regulating cytokine storm.

The inosine may reduce SARS-COV-2 induced pathological changes of lung tissue and inhibit the secretion of cytokine IL-6. In the process of SARS-COV-2 induced cytokine storm (CRS), the IL-6 is one of the most critical cytokines, and severity of the CRS is closely related to the increase in IL-6. Therefore, the control of the IL-6 is conducive to the control of the cytokine storm; the inosine may inhibit high expression of SARS-COV-2 induced IL-6 to effectively control SARS-COV-2 caused inflammatory lung injury, further substantially increasing the percent survival of critically ill patients with SARS-COV-2.

To sum up, the present disclosure has the following beneficial effects:
1. the inosine can downregulate high expression of SARS-COV-2 induced IL-6 to effectively control SARS-COV-2 caused inflammatory lung injury, further substantially increasing the percent survival of critically ill patients with SARS-COV-2;
2. the inosine brings hope to severe and critically ill patients with COVID-19 and their families, and provides a new idea for treating severe and critically ill patients with COVID-19.

### BRIEF DESCRIPTION OF THE DRAWINGS

FIG. 1 is a statistical graph of experimental data showing that inosine in the example of the present disclosure can increase the percent survival of lethal animal models by COVID-19;

FIG. 2 is a statistical graph of experimental data showing that inosine in the example of the present disclosure can promote regain of weight of SARS-COV-2-infected animals;

FIG. 3 is a schematic diagram of experimental results showing that inosine in the example of the present disclosure can achieve effective control and recovery of inflammatory lung injury in SARS-COV-2-infected animals; and

FIG. 4 is a schematic diagram of experimental results showing that inosine in the example of the present disclosure can inhibit levels of SARS-COV-2 induced IL-6.

### DETAILED DESCRIPTION OF THE EMBODIMENTS

The present disclosure will be further described in detail below with reference to drawings and an example:

Example: use of a drug in the treatment of COVID-19, where the drug was inosine. The inosine was used to inhibit the level of IL-6, thereby inhibiting SARS-COV-2. The formulations of inosine included one or more selected from the group consisting of tablets, capsules, oral liquid preparations, emulsions, and injections.

The inosine could substantially increase the percent survival of critically ill patients with SARS-COV-2 infection. The detection of the example had the following results.

A. The inosine could increase the percent survival of lethal animal models by COVID-19 and promote regain of weight of SARS-COV-2-infected animals.

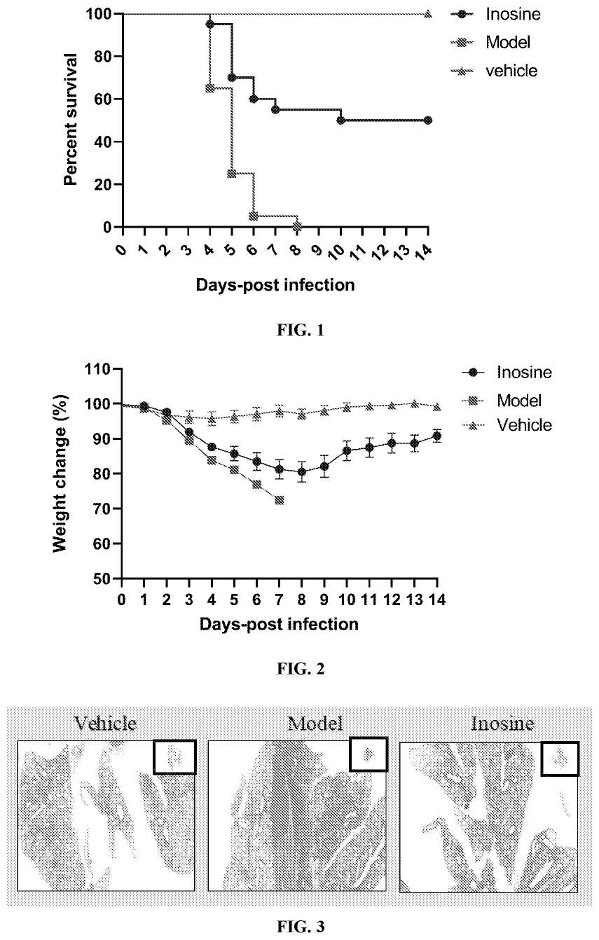

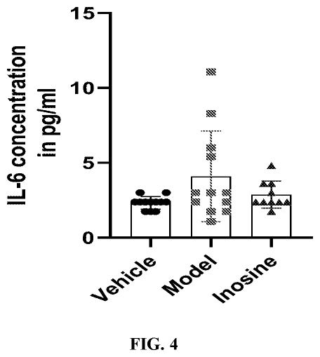

As shown in FIGS. 1 to 2, the inosine could increase the percent survival of mice. Research results showed that SARS-COV-2-infected Balb/c mice (model group) died of multi-organ inflammation due to virus infection on days 4-7, with significant weight loss. After preventively administration of inosine (inosine group), it was observed that the survival time of the Balb/c mice was significantly prolonged, the mice had lower weight loss, and their body weights regained gradually on day 8 after infection.

B. The inosine could reduce SARS-COV-2 induced pathological changes of the mouse lung tissue.

As shown in FIG. 3, the inosine could reduce SARS-COV-2 induced pathological changes of the mouse lung tissue. The effect of inosine on pathological changes of lung tissue was observed by hematoxylin and eosin (H&E) staining. Results showed that petechial or splinter hemorrhage, massive neutrophils infiltration, and pulmonary interstitial edema were observed in the SARS-COV-2 infection group (model group) compared with the blank control group (vehicle group); while in the oral inosine administration group (inosine group), the lung tissues were structurally intact, alveolar space was clear, no congestion was noted in the pulmonary alveolar wall, and inflammatory infiltration was significantly improved compared with the SARS-COV-2 infection group.

C. The inosine could inhibit the secretion of cytokine IL-6.

In the process of cytokine storm (CRS), IL-6 is one of the most critical cytokines, and severity of the CRS is closely related to the increase in IL-6. In the cytokine storm induced by SARS-COV-2, IL-6 plays an important role. In order to determine whether the inosine could inhibit the secretion of IL-6, the level of cytokine IL-6 in mouse serum was determined by ELISA. Results showed that the inosine (inosine group) could inhibit the high expression of SARS-COV-2 induced IL-6, and control of IL-6 was conducive to the control of the cytokine storm.

Conclusion: the inosine could effectively control SARS-COV-2 caused inflammatory lung injury by inhibiting the level of IL-6, and substantially increase the percent survival of critically ill patients with SARS-COV-2.

In the above example of the present disclosure, the drug can downregulate high expression of SARS-COV-2 induced IL-6 to effectively control SARS-COV-2 caused inflammatory lung injury, further substantially increasing the percent survival of critically ill patients with SARS-COV-2. The drug brings hope to severe and critically ill patients with COVID-19 and their families, and provides a new idea for treating severe and critically ill patients with COVID-19.

This specific example is merely an explanation of the present disclosure, but it does not limit the present disclosure. Those skilled in the art can make modifications without creative contribution to the present example as needed after reading this specification, but these modifications are protected by patent law as long as they fall within the claimed scope of claims herein.

What is claimed is:

1-6. (canceled)

7. A method of prevention and/or treatment of Corona Virus Disease 2019 (COVID-19), comprising using inosine or a drug comprising inosine.

8. The method according to claim 7, wherein the treatment of the COVID-19 with the inosine or the drug is conducted by inhibiting the secretion of cytokine interleukin-6 (IL-6) and/or alleviating severe acute respiratory syndrome coronavirus 2 (SARS-COV-2) caused inflammatory lung injury.

9. The method according to claim 7, wherein the treatment of the COVID-19 comprises increasing the percent survival of treating severe and/or critically ill patients with the COVID-19.

10. The method according to claim 7, wherein a formulation of the inosine comprises one or more selected from the group consisting of tablets, capsules, oral liquid preparations, emulsions, and injections.

11. The method according to claim 7, wherein a formulation of the drug comprises one or more selected from the group consisting of tablets, capsules, oral liquid preparations, emulsions, and injections.
